(19) United States
(12) Patent Application Publication
(10) Pub. No.: US 2018/0206778 A1
(43) Pub. Date: Jul. 26, 2018

(54) ATROPHIC ACNE SCAR RISK ASSESSMENT TOOL

(71) Applicant: NESTLÉ SKIN HEALTH, Lausanne (CH)

(72) Inventors: Jerry TAN, Windsor (CA); Brigitte DRENO, Nantes Cedex (FR); Harald GOLLNICK, Magdeburg (DE); Sewon KANG, Baltimore, MD (US); Alison LAYTON, Harrogate (GB); James LEYDEN, Malvern, PA (US); Diane THIBOUTOT, Hersey, PA (US); Vicente Torres LOZADA, Estado De Mexico (MX); Jonathan GUILLEMOT, Cumbaya (EC)

(21) Appl. No.: 15/875,508

(22) Filed: Jan. 19, 2018

Related U.S. Application Data
(60) Provisional application No. 62/448,927, filed on Jan. 20, 2017.

(51) Int. Cl. A61B 5/00; G16H 10/20; G16H 20/10

(52) U.S. Cl. CPC A61B 5/441; G16H 20/10; G16H 10/20

(57) ABSTRACT

A method is described for detecting and treating an individual subject having an increased risk for acne scarring.

### CROSS-REFERENCE TO PRIOR APPLICATIONS

This application claims priority under 35 U.S.C. § 119 to U.S. Provisional Patent Application No. 62/448,927, filed Jan. 20, 2017, hereby expressly incorporated by reference in its entirety and assigned to the assignee hereof.

### FIELD

The present disclosure relates to tools and methods for the detection, assessment, and treatment of acne scars.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

Currently, there are two types of skin classification methods used in dermatology. The first is based on the skin's reactivity to the sun and was developed by Thomas B. Fitzpatrick in the 1960's. The Fitzpatrick scale has become ubiquitous in dermatology. However, the Fitzpatrick scale only provides general guidelines for sunscreen in an attempt to prevent and/or combat sun damage. The second classification method is the Glogau photo-aging scale, which divides skin into four categories based on the amount of wrinkles that are present. Neither the Fitzpatrick scale nor the Glogau photo-aging scale address or even attempt to classify or assess acne or any scarring caused by acne.

Acne is a chronic disease that predominantly afflicts young adults from about the age of 11 years old.¹ Acne peaks in young adults around 16 years old, and persists in some to the their 20s and 30s.^{2,3} Acne is associated with significant psycho-social burden, including, but not limited to, depression, anxiety, deficits in quality of life, which is greater than in patients with chronic disabling asthma, epilepsy, diabetes, or arthritis.⁴⁻⁶ Treatments comprise topical agent monotherapies including retinoids, antibiotics, benzoyl peroxide, as monotherapies or combination, and systemic agents including antibiotics, antiandrogens and isotretinoin.⁷⁻⁹

Acne is often associated with the development of scars. The scars are usually in moderate or severe inflammatory forms, but can also present as a mild form as well.¹⁰⁻¹¹ In patients with moderate acne, scars can form from papules (inflammatory lesions) or post-inflammatory lesions with only a third resolving within six months.¹² Thus, there is a link between early acne management and scar prevention. Although the research is in its infancy, acne scarring is often associated with burdensome psycho-social consequences and deficits in the quality of life.¹³⁻¹⁴ Acne scars are also associated with decreased self-confidence, including perception of decreased employability and embarrassment, and is often negatively perceived by society.¹⁵⁻¹⁶ These further negative effects have been recognized by the American Academy of Dermatology and by practitioners around the world, and further reinforces the need for early and effective treatments.

Management of atrophic acne scars include dermal corrective procedures such as filler, dermabrasion, micro-needling, or laser resurfacing.¹⁷ However, such procedures present potential risks including adverse events, high costs, and uncertain efficacy.¹⁸ Acne scarring is often avoidable if managed early and effectively. Thus, there is a need for a new and innovative assessment tool to assess the risk of atrophic acne scars in patients in need thereof and initiate early treatment to prevent or minimize the acne scars.

### SUMMARY

Applicants have found an exemplary tool and method for detecting and treating an increased risk for acne scarring. In exemplary embodiments, the method comprises measuring an increased risk factor for acne scarring in a patient in need thereof. The increased risk for acne scarring is measured and quantified according to the risk factor(s) for acne scarring. The risk factors generally fall in one of four categories: (1) severity of acne, (2) a family history of acne scarring, (3) the duration of time of acne, and (4) a behavior of squeezing and picking acne lesions. If a patient is diagnosed with an increased risk factors for acne scarring, an anti-acne active is administered to reduce or prevent (or substantially inhibit) acne scarring.

The aim of the present disclosure is the development of a tool for assessing the risk of acne scars, in particular atrophic acne scars. Applicants conducted a systematic literature review of clinical risk factors for acne scars, a Delphi-like survey of dermatological experts in acne and secondary data analysis to produce an evidence-based risk assessment tool. The tool was assessed both with a sample of young adults with and without scars and was assessed via a database cross-validation.

A self-administered tool for risk assessment of developing atrophic acne scars in young adults was developed. It is a readily comprehensible and practical tool for population education and for use in medical practices. The four risk factors—worst ever severity of acne, duration of acne, family history of atrophic acne scars and lesion manipulation behaviors—provide a dichotomous outcome: lower versus higher risk of developing scars. The tool, thereby, categorizes nearly two thirds of the population correctly, with sensitivity of 82% and specificity of 43%.

The present tool was developed as a response to current challenges in acne scar prevention. A potential benefit is to encourage those at risk to self-identify and to seek active intervention of their acne. In clinical practice, it is expected that this tool will help clinicians identify patients at risk of atrophic acne scarring and underscore their requirement for rapid and effective acne treatment.

### BRIEF DESCRIPTION OF THE DRAWINGS

FIG. 1 depicts an embodiment of a method of detecting an increased risk for acne lesions.

FIG. 2 depicts one embodiment for diagnosing an increased risk for acne lesions.

FIG. 3 depicts a self-assessment tool for measuring an increased risk for acne lesions.

FIG. 4 depicts a standardization table for the severity of acne.

FIG. 5 depicts atrophic acne scarring in patients.

### DETAILED DESCRIPTION

Further aspects, features and advantages of the exemplary embodiments will become apparent from the detailed description which follows.

The patents, published applications and scientific literature referred to herein establish the knowledge of those with skill in the art and are hereby incorporated by reference in their entireties to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of a method, the term “comprising” means that the method includes at least the recited steps, but may include additional steps. When used in the context of a composition, the term “comprising” means that the composition includes at least the recited features or components, but may also include additional features or components.

The terms “consists essentially of” or “consisting essentially of” have a partially closed meaning, that is, they do not permit inclusion of steps or features or components which would substantially change the essential characteristics of a method or composition; for example, steps or features or components which would significantly interfere with the desired properties of the compounds or compositions described herein, i.e., the method or composition is limited to the specified steps or materials and those which do not materially affect the basic and novel characteristics of the method or composition.

The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Technical and scientific terms used herein have the meaning commonly understood by one of skill in the art to which the present description pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10th Ed., McGraw Hill Companies Inc., New York (2001).

Applicants have discovered a tool for risk assessment of developing atrophic acne scars in young adults. In a preferred embodiment, the tool is self-administered by a subject in need thereof. In an alternative embodiment the tool is used in a medical practice by a medical professional. In one embodiment the tool comprises measuring at least one risk factor, wherein the risk factor includes, but is not limited to, the worst ever severity of acne, duration of acne, family history of acne scars and lesion manipulation behaviors. In an exemplary embodiment, the tool provides a dichotomous outcome. In a preferred embodiment, the dichotomous outcome determines lower versus higher risk of developing atrophic acne scars. The tool is able to categorize about two thirds of the population correctly, with sensitivity of about 82% and specificity of about 43%.

Applicants have conducted a rigorous and comprehensive process comprising: a systematic literature review, survey based on the Delphi method of dermatological experts in acne and secondary data analysis, using a database of more than 1,100 subjects. The tool was assessed both with a sample of young adults with and without atrophic scars and cross-validated against a real-world database and showed strong statistical robustness. During the qualitative assessment, the tool was well-received and was meaningful to interviewees.

Atrophic acne scarring can occur even in subjects with mild acne. When subjects display a mild severity of acne but combine other risk factors such as family history of acne scarring and duration of acne longer than one year, doctors should treat those subjects with the same approach as subjects with moderate acne. The psychological impact of acne scars is burdensome and atrophic acne scar management is complex. It is often associated with additional costs and could induce side effects. Early and effective treatment of acne is the most efficient means of preventing or substantially inhibiting acne scars. The present tool was conceived as a response to current challenges in atrophic acne scar prevention/reduction as it may facilitate increased awareness of those at higher risk of atrophic acne scarring.

A specific benefit is to encourage those at risk to self-identify and to seek active intervention of their acne. In clinical practice, we expect this tool may help clinicians identify subjects at risk of atrophic acne scarring and underscore their requirement for rapid and effective acne treatment.

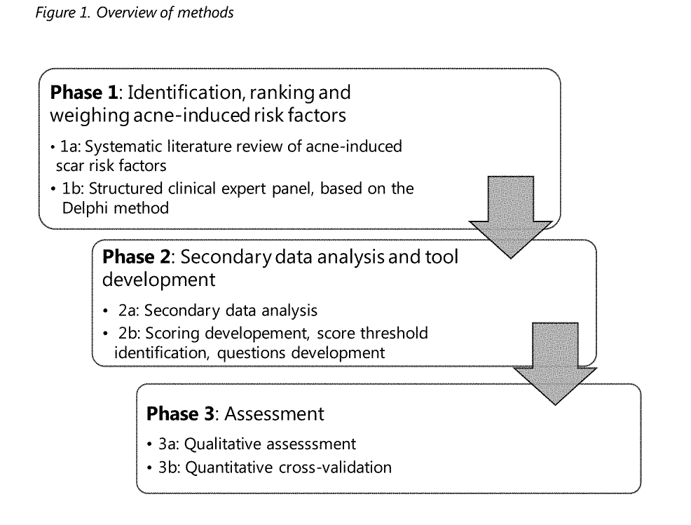

In a preferred embodiment, the tool comprises three phases: (1) identification, ranking and weighing risk factors associated with acne scarring from a literature review, a Delphi-like expert panel; (2) tool development; and (3) assessment of operating characteristics, as shown in FIG. 1.

In one embodiment, rank and weigh risk factors associated with atrophic acne scarring are determined by (1a) a systematic review of risk factors associated with acne scarring, (1b) a structured clinical expert consensus to complete and rank risk factors associated with acne scarring, and (2a) a secondary data analysis to weigh risk factors were implemented. All phases can be overseen by a scientific committee composed of dermatologists, statisticians and qualitative researchers.

Phase 1a: Systematic Review

A systematic review regarding risk factors associated with development of acne scars is shown in Table 1. Search terms for MEDLINE and EMBASE databases, respectively searched via Pubmed and Ovid, are in Supplementary Material 1.

TABLE 1
Systematic literature review methodology and search results

| | |
|---|---|
| Search details | |
| Databases searched | MEDLINE (via PUBMED) |
| | EMBASE (via EMBASE) |
| Limits | Population: Patients with acne scars |
| | Intervention: No restriction |
| | Comparator: No restriction |
| | Outcomes: Factors associated with acne scars |
| | Study Type: Clinical trials, observational studies, |
| | reviews |
| | Dates and geography: No restriction |
| Hand searches | Dermatology organizations, relevant conference |
| | abstracts/posters (from 2012) |
| Inclusion criteria | Conference abstracts of interest: published 2012-2016 |
| | Study type of interest: clinical trials, observational |
| | studies and reviews |
| | Population of interest: Patients with acne scars |
| | Outcome of interest: Risk factors for acne scarring |
| | Locations of interest: no limits |
| Exclusion criteria | Duplicate, not in the language of interest (English), |
| | abstract that is reported elsewhere |
| Search results | |
| Number of references | n = 2632 |
| identified through the | |
| systematic literature searches | |
| Number of references | n = 2 |
| identified through the hand | |
| searches | |
| Number of articles included | n = 17 |
| in the systematic review | |

Phase 1b: Structured Clinical Expert Panel

Applicants prepared a structured expert panel to (1) assess the relevance of potential risk factors identified from the literature review and (2) ranked in order of importance. An online process, inspired by the Delphi method was undertaken.¹⁹⁻²⁰ Panelists were clinical experts identified on the basis of their scientific contribution to the field of acne (publication, keynotes and clinical trials). A survey questionnaire was developed and submitted to panelists on an iterative basis on three occasions. The latter two were complemented by qualitative summaries of previous questionnaire responses.

The relevance of factors was assessed via a qualitative analysis of results from the third round of the structured expert panel. Potential items were organized in three relevance categories: “more relevant risk factors”, “possibly relevant risk factors” and “less relevant risk factors”. The final ranking was established at the end of the third round of the structured expert panel. After each round, the median ranking of each potential risk factor was calculated and communicated in the following round to all participating panelists. The list of the most relevant risk factors was retained at the end of this phase.

Phase 2a: Secondary Data Analysis

To weigh risk factors for atrophic acne scars retained from the two previous phases, a quantitative analysis of secondary data was conducted. Patients with and without atrophic scars were compared and odds ratios (OR) for the retained risk factors—presented subsequently as part of the findings—were calculated using logistic regression. Based on the latter, a predictive tool was developed to address individual probability of developing atrophic acne scars. A receiver operating curve (ROC) curve was plotted and the most appropriate threshold was selected by the scientific committee based on clinical relevance.²¹ Descriptions of the database as well as the methodological approach to statistical analyses are presented in Supplementary Material 3.

Phase 2b: Tool Development

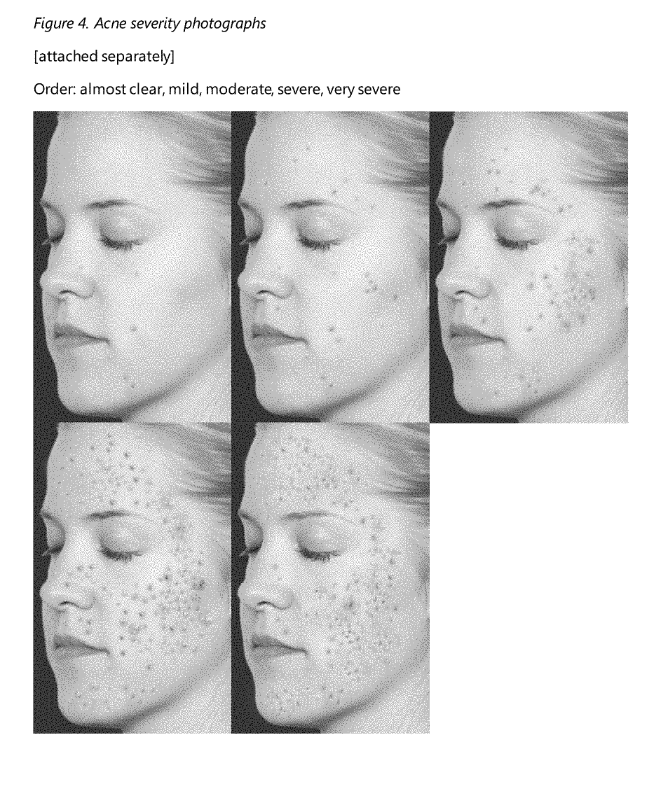

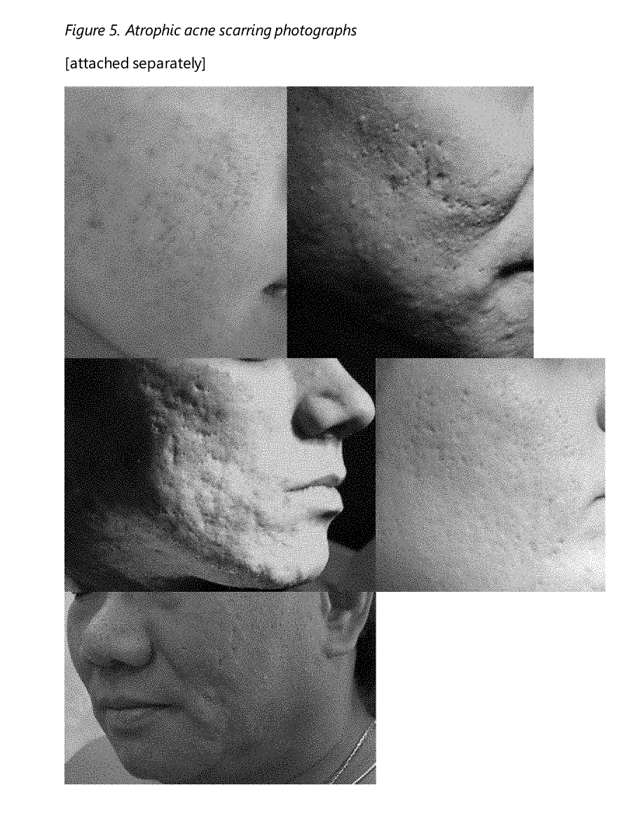

Retained risk factors were transformed into lay language questions. With the findings of the secondary data analysis, a score was associated with each possible answer and the threshold to categorize tool respondents into lower or higher risk of developing atrophic acne scars was calculated. The primary aim was risk detection. An algorithm leading to the outcome depending on the answer on each answer was developed. The questionnaire was accompanied with two sets of photographs. The first one, presented in FIG. 4, was designed to help respondents identify their severity of acne instead of using a literal description. The second set of photographs, presented in FIG. 5, was designed to support respondents understand what is meant by atrophic acne scars.

Exemplary Tool Characteristics

Based on other comparable tools, in particular for the prevention of melanoma, the tool can be self-administered, to require just a few minutes to complete, to be usable both with pen and paper and online and to provide a simple and easily comprehensible dichotomous outcome: lower or higher risk of developing atrophic acne scars. In an exemplary embodiment the tool targets people with active acne in particular at acne onset.²²⁻²⁴

Phase 3a: Qualitative Tool Assessment

The qualitative assessment aimed to test the outcome of the questionnaire and to assess the comprehensibility of the tool phrasing. For feasibility purposes, Applicants interviewed 10 persons and the target population was restricted to young adults aged 18 to 25, with or without atrophic acne scars and without active acne. Ethnic diversity was sought to test the usability and acceptability of the model used in the photographs. The methodological approach followed strict ethical standards and was approved by an internal ethics committee. After informed consent was obtained, interviewees were asked to complete the tool as they would have when their acne was at its worst. Participants subsequently responded to a semi-structured interview.

Interviews were audio recorded and followed a topic guide, which included comprehensibility and feasibility of the questionnaire and its outcome, acceptability and usefulness of the tool and the appropriateness of the photograph models selected to illustrate the severity of acne and examples of atrophic acne scarring. Portrait photographs of each interviewee were captured, then anonymized. Photographs were assessed by a clinical dermatologist to establish the presence and degree of atrophic acne scarring. The tool outcome for each respondent was compared to the existence of atrophic acne scars established by the interviewee and by the dermatologist.

Phase 3b: Quantitative Cross-Validation

Cross validation of the tool was undertaken with a pre-existing acne survey database. The proportions of true and false positives and negatives were computed (Supplementary Material 4).

Systematic Review of Acne Scar Risks

Searches yielded 2632 citations, of which 17 fulfilled all inclusion criteria, as presented in Table 1. Ten publications were observational studies while seven were literature reviews. The PRISMA diagram and the exhaustive list of studies can be found in Supplementary Material 1.

Seventeen factors associated with the development of acne scarring from the literature were identified. Identified risk factors were: acne inflammation, severity of acne, duration of untreated acne, duration of acne, family history of acne scarring, gender, ethnicity, smoking, duration of acne therapy, treatment compliance, treatment fatigue, age, patient demographics, seborrhea, body mass index, relapse after treatment, and picking and squeezing behavior. The results of the review indicate that scarring develops to some degree in the majority of acne patients, but did not conduct any quantitative analysis to determine the relative weight for risk factors.¹⁰

There is a paucity of studies examining risk factors for acne scarring, in particular studies providing a quantitative assessment of the risk factors, none of which used methods proposed in the study presented in this application. The first element in the evolution of an acne scar is evolution of acne itself.²⁵ Acne severity and inflammation of lesions tends to lead to increased scarring. Evidence points towards a causal association leading to scarring between persisting antigen presentation and delayed and protracted inflammation. Strong and short inflammation with marked T-cell reaction was associated with less scarring.²⁶ Studies indicate that treatment to prevent, reduce and/or substantially inhibit worsening of acne symptoms and to shorten disease duration contribute to the prevention, reduction and/or substantial inhibition of scarring. The paucity and inadequacy of studies prompted the development of the following study phases.

Structured Expert Panel

Fifteen recognized clinical experts participated in the structured expert panel. Panelists were practitioners from eight countries over three continents. Further characteristics of participating clinical experts are available in Supplemental Material 2. Findings related to the relevance and ranking of potential risk factors for atrophic acne scarring are displayed in Table 2. Items considered as more relevant risk factors for atrophic acne scarring were severity of acne (median rank: 1), inflammation of acne lesions (median rank: 2), duration of acne (median rank: 3), family history of acne scarring (median rank: 5), squeezing and picking behaviors (median rank: 5) and patient history of acne scarring (median rank: 5). The rate of agreement—the proportion of experts agreeing on the ranking (+/−1 rank) at the end of the third round—was high and illustrated a large consensus level. It was on average 0.75 and ranged from 0.33 (seborrheic skin) to 1 (Severity of acne and inflammation of lesions). Findings related to the relevance of factors and ranking of factors were coherent and supported each other factors, as top ranked factors were generally those categorized as more relevant factors by panelists. Data related to the relevance of risk factors from the third round of the structured expert panel can be found as Supplemental Material 2.

TABLE 2
Relevance and ranking of risk factors according to panelists in
the structured expert panel

| | Relevance | |
|---|---|---|
| Potential risk factors | of factors | Ranking* |
| Severity of acne | More relevant | 1 |
| Inflammation of acne lesion | | 2 |
| Duration of acne | | 3 |
| Family history of acne scarring | | 5 |
| Squeezing/picking behaviors | | 5 |
| Patient history of acne scarring | | 5 |
| Patient compliance | Possibly relevant | 8 |
| History of relapse after treatment | | 9 |
| Location of acne lesions | | 9 |
| Skin preparation (comedone/cyst removal) | | 10 |
| Patient history of scarring (not related to | | 12 |
| acne) | | |
| Treatment type | | 12 |
| Seborrheic skin type | Less relevant | 14 |
| Ethnicity | | 17 |
| Gender | | 18 |
| Smoking | | 18 |
| Geographical location | | 18 |
| Body mass index | | 18 |

Although the opportunity for panelists to introduce alternative risk factors was given within the questionnaire, none were proposed. The structured expert panel provided a ranked list of risk factors around which consensus was met, which supported the development of the following phase aiming to assess the relative weights of risk factors in the development of atrophic acne scars.

Secondary Data Analysis

“Severity of acne” was found to be the strongest risk factor with an OR of 3.68, followed by “family history of acne scarring” with an OR of 2.14. Risk factors “duration of acne” and “squeezing and picking behaviors” displayed similar OR, respectively at 1.64 and 1.70. All four risk factors were found to be significantly associated with increased atrophic scarring with lower confidence intervals above 1. As “inflammation of acne lesions” was judged unclear to the tool target population and as it was combined with “severity of acne” in the data set, the factor “inflammation of lesions” was combined with the factor “severity of acne”. The scientific committee decided to leave aside the risk factor “patient history of acne scars” as the tool was targeting primarily young adults who do not yet have atrophic scars. Further details can be found in Supplementary Material 3.

Table 3. Risk Factors Odds Ratios

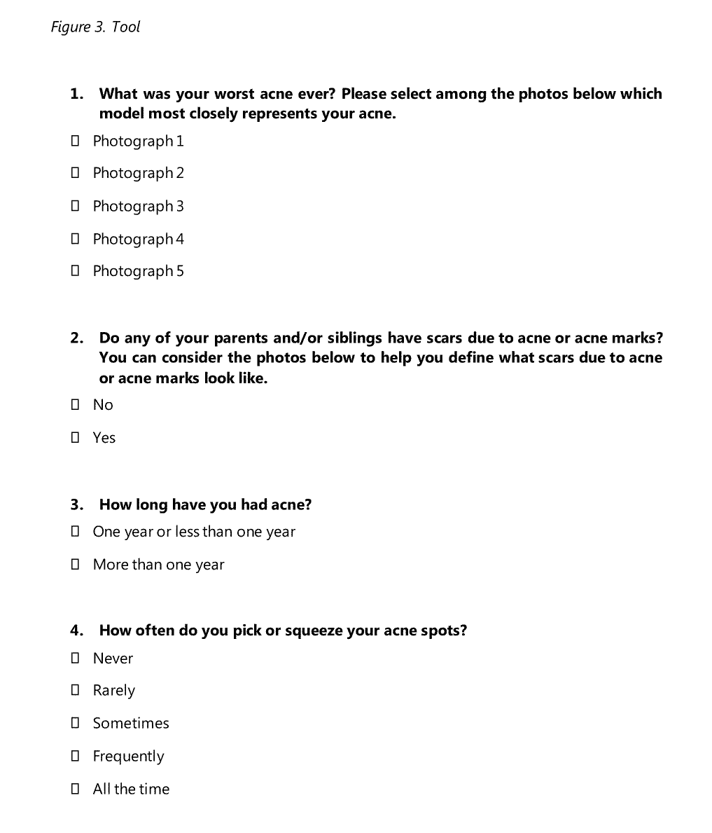

Tool questions are presented in FIG. 3. Based on ORs presented above, scores associated with all possible answers were calculated and are presented in Table 4.

TABLE 4
Score

| | | Point |
|---|---|---|
| Risk factor | Answer | added to score |
| Severity of acne | Photograph 1 (almost clear) | 0 |
| | Photograph 2 (mild) | 0 |
| | Photograph 3 (moderate) | 0 |
| | Photograph 4 (severe) | 8.90 |
| | Photograph 5 (very severe) | 8.90 |
| Family history of acne | No | 0 |
| scarring | Yes | 4.60 |
| Duration of acne | Less than one year and one year | 0 |
| | More than one year | 3.46 |
| Squeezing and picking | Never | 0 |
| behaviors | Rarely | 0 |
| | Sometimes | 0 |
| | Frequently | 3.05 |
| | All the time | 3.05 |

| | |
|---|---|
| Threshold to exceed to be categorized as “higher risk” | 3.95 |

Table 4. Severe to very severe acne is associated with 8.90 points; having siblings or parents with acne scars leads to the addition of 4.60 points, while having had acne for more than one year and picking or squeezing lesions frequently or all the time lead to 3.46 and 3.05 points respectively. All other answers were assigned zero points.

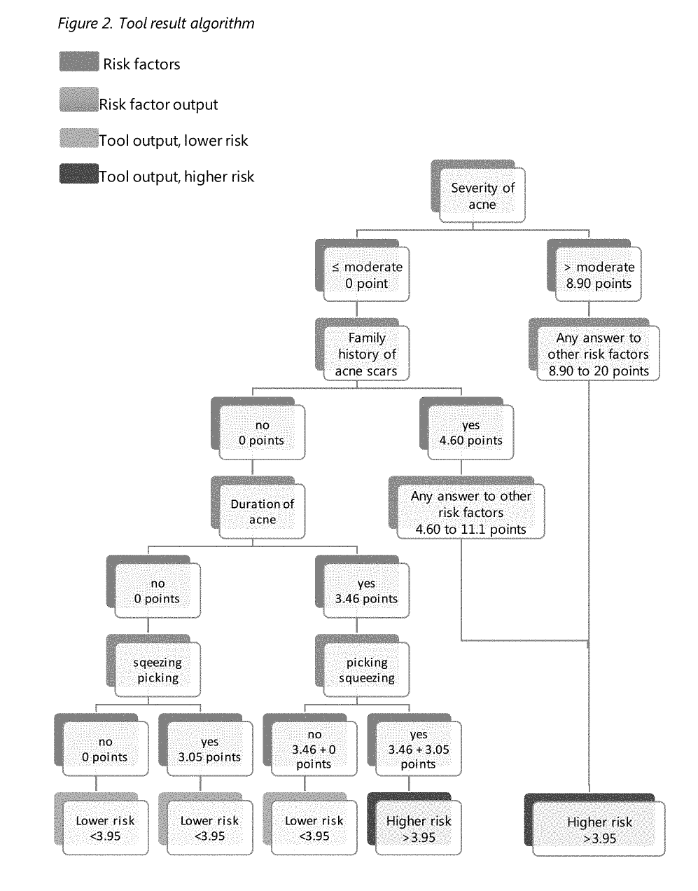

The threshold categorizing higher risk of developing atrophic acne scars was established at 3.95 with higher sensitivity judged of greater importance—with an expected reduction of specificity. Consequently, to be categorized as higher risk, respondents must either fulfill the risk factor associated with severity of acne, the risk factor associated with family history of acne scars or both risk factors related to duration of acne and lesion manipulation behavior. The algorithm, presented in FIG. 2, was designed to simplify the outcome calculations and make possible the use the tool both with pen and paper and online. Further details are included in Supplementary Material 3.

Tool Assessment

To qualitatively assess the tool, ten young adults were interviewed. Half were females, all resided in England and the median age was 21.5 years, ranging from 20 to 24 years. Interviewees' skin tones were reported according to the Fitzpatrick scale and data is shown in Table 5.²⁸ Three had atrophic acne scars according to the dermatologist's assessment, which differed from the interviewee's self-assessment in two cases. Six interviewees were appropriately categorized while there were zero false negative and four false positives. Findings are displayed in Table 5 and the data table, including the interviewees' past severity of acne, is available in Supplementary Material 4.

TABLE 5
Overview of qualitative findings

| | | Scar assessment | | Tool outcome | | Tool |
|---|---|---|---|---|---|---|
| Interviewees | Ethnicity* | Self | Dermatologist | Score | Category | evaluation |
| 1 | II | No | No | 4.60 | Higher risk | False positive |
| 2 | IV | Yes | No | 3.46 | Lower risk | Appropriate |
| 3 | I | Yes | Yes (minimal/mild) | 11.11 | Higher risk | Appropriate |
| 4 | III | No | No | 0 | Lower risk | Appropriate |
| 5 | III | No | No | 11.11 | Higher risk | False positive |
| 6 | II | Yes | Yes (minimal/mild) | 8.06 | Higher risk | Appropriate |
| 7 | IV | Yes | No | 3.46 | Lower risk | Appropriate |
| 8 | VI | No | No | 8.06 | Higher risk | False positive |
| 9 | V | Yes | Yes (moderate) | 11.11 | Higher risk | Appropriate |
| 10 | I | No | No | 8.06 | Higher risk | False positive |

The cross-validation with the database used for the secondary data analysis to the tool, resulted in 61% of the patients appropriately categorized, i.e., patients with atrophic scars categorized as higher risk and patients without atrophic scars as lower risk; and 39% of patients inappropriately categorized: 57% of positives were false positive and 18% of negatives were false negative.

The term “acne mark” was added to “scars due to acne” as it was more often used and comprehensible by respondents. The photograph of the female model used to represent the severity of acne was well received and respondents had no hesitation to select their worst ever acne severity, although three respondents would have preferred a model displaying their skin tone. However, respondents also indicated that acne lesions were more clearly identifiable on fair skin. Overall, the questionnaire was comprehensible and well received. The tool outcome was clear and meaningful to the respondents.

Supplementary Materials

Supplementary Material 1.

Supplementary material regarding the systematic literature review

Search terms from MEDLINE and MEDLINE-in-Process via Pubmed

| # | Search terms | Results |
|---|---|---|
| 1 | ((acne[Title/Abstract] OR acne[MeSH]) AND | 1,106 |
| | scar*[Title/Abstract]) | |

Search Terms from EMBASE Via Ovid

| # | Search terms | Results |
|---|---|---|
| 1 | acne.ti,ab. | 18,879 |
| 2 | Scar*.ti,ab. | 158,735 |
| 3 | #1 and #2 | 1,642 |

List of Included Studies

| # | Included References |
|---|---|
| 1 | Layton, A M; Henderson, C A; Cunliffe, W J. 1994. A clinical evaluation of acne |
| | scarring and its incidence. Clinical and Experimental Dermatology. 19(4); |
| | 303-308. |
| 2 | Layton, A M; Seukeran, D; Cunliffe, W J. 1997. Scarred for Life? Dermatology. |
| | 195(Suppl. 1); 15-21 |
| 3 | Hayashi, N; Miyachi, Y; Kawashima, M. 2015. Prevalence of scars and “mini- |
| | scars”, and their impact on quality of life in Japanese patients with acne. |
| | Journal of Dermatology. 42(7); 690-6. |
| 4 | Basak, S A; Zaenglein, A L. 2013. Acne and Its Management. Pediatrics in |
| | Review. 34(11); 479-97. |
| 5 | Goodman, G J. 2000. Management of post-acne scarring. What are the options |
| | for treatment? American journal of clinical dermatology. 1(1); 3-17. |
| 6 | Rajar, U D; Majeed, R; Sheikh, F; Sheikh, I; Siddique, A A; Kumar, S. 2009. |
| | Scarring in acne patients--a study done at Isra University Hyderabad. The |
| | Journal of the Pakistan Medical Association. 59(8); 525-7. |
| 7 | Goodman, G J. 2001. Post-acne scarring: A short review of its pathophysiology. |
| | Australasian Journal of Dermatology. 42(2); 84-90 |
| 8 | Goodman, G J. 2006. Acne--natural history, facts and myths. Australian family |
| | physician. 35(8); 613-6. |
| 9 | Holland, D B; Jeremy A H; Roberts, S G; Seukeran, D C; Layton, A M; Cunliffe, W J. |
| | 2004. Inflammation in acne scarring: a comparison of the responses in lesions |
| | from patients prone and not prone to scar. Br J Dermatol. 150(1); 72-81. |
| 10 | Rivera, A E. 2008. Acne scarring: A review and current treatment modalities. |
| | Journal of the American Academy of Dermatology. 59(4); 659-76. |
| 11 | Tan, J K.; Tang, J; Fung, K; Gupta, A K; Richard Thomas, D; Sapra, S; Lynde, C; |
| | Poulin, Y; Gulliver, W; Sebaldt, R J. 2010. Development and validation of a Scale |
| | for Acne Scar Severity (SCAR-S) of the face and trunk. J Cutan Med Surg. 14(4); |
| | 156-60. |
| 12 | Jacob, C I; Dover, J S; Kaminer, M S. 2001. Acne scarring: a classification system |
| | and review of treatment options. J Am Acad Dermatol. 45(1); 109-17. |
| 13 | Tuan, T L; Nichter, LS. 1998. The molecular basis of keloid and hypertrophic |
| | scar formation. Mol Med Today. 4(1); 19-24. |
| 14 | Holland, D B; Jeremy, A H. 2005. The role of inflammation in the pathogenesis |
| | of acne and acne scarring. Semin Cutan Med Surg. 24(2); 79-83 |
| 15 | Czernielewski, J; Dreno, B; Layton, A; Bettoli, V; Torres Lozada, V; Sewon, K. |
| | 2015. Evaluation of the prevalence, risk factors, clinical characteristics, and |
| | burden of acne scars among active patients in Brazil, France and the USA. |
| | WCD 23rd Annual Meeting (Abstract 2982723). |
| 16 | Kadiyala, P; Layton, A; Wartburton, K; Bhutani, R; Fryat, E; Allgar, V. 2015. |
| | Predicting factors influencing facial atrophic scarring in acne. 96th Annual |
| | Meeting - British Association of Dermatologists. |
| 17 | Bhutani, R; Kadiyala, P; Fryatt, E; Layton, A M. 2015. Smoking and Atrophic |
| | Facial Scarring in Acne. 96th Annual Meeting - British Association of |
| | Dermatologists. |

Supplementary Material 2.

Supplementary material related to the structured expert panel

Overview of Panelists' Characteristics

| Items | Findings |
|---|---|
| Practice | Private practice (n = 8), public hospital (n = 6, |
| | private hospital (n = 2)* |
| Median number of patients with acne | 0-300 [90] |
| overseen per month (range [median]) | |
| Countries of practice | USA (n = 6), Germany (n = 2), Canada (n = 1), |
| | France (n = 1), Italy (n = 1), Mexico (n = 1), UK |
| | (n = 1), Singapore (n = 1), South Korea (n = 1) |
| Median number of publications in acne | 5-65 [16] |
| in the last five years (range [median]) | |
| Median number of keynotes in the last | 3-79 [15] |
| five years (range [median]) | |
| Median number of randomized | 0-30 [8] |
| controlled trials in the last five years | |
| (range [median]) | |

*One panelist practiced both within a private practice and in a public hospital

Findings from the Third Round of the Structured Expert Panel: Relevance of Risk Factors

| Potential risk factors | Relevance data (qualitative summary) |
|---|---|
| Severity of acne | Though not true in all patients, the severity of acne is |
| | closely linked to the development of atrophic acne scars. |
| Inflammation of acne lesion | Higher degrees of Inflammation is a risk factor for acne |
| | atrophic scarring. Patients with more inflamed lesions are |
| | more likely to scar. |
| Duration of acne | The duration of acne is a relevant risk factor. Generally - |
| | not systematically—the longer patients have acne, the |
| | more likely they are to develop scars. Nevertheless, |
| | patients with long-lasting acne may display little or no |
| | scarring. |
| Family history of acne | Family history of acne scarring is a likely risk factor for |
| scarring | scarring, as propensity to scar has a strong genetic |
| | background. |
| Squeezing/picking | Acne-related behaviors are risk factors for acne atrophic |
| behaviors | scars. These behaviors aggravate inflammation and cause |
| | destruction of the dermis. The combined contributions |
| | made by the cumulative effect arising from prolonged |
| | acne inflammation, and the mechanical damage arising |
| | from picking/squeezing, remain unclear. |
| Patient history of acne | History of scarring related to acne is a risk factor for acne |
| scarring | scars, particularly in patients prone to scarring. |
| Patient compliance | Poor compliance to treatments leads to delayed response |
| | causing relapse and further inflammation and in some |
| | cases treatment failure. There are varying degrees of |
| | compliance which may or may not have an impact on |
| | treatment efficacy, for instance; a patient who adheres to |
| | the treatment some of the time achieving a reduction in |
| | inflammation may experience relatively less scarring as |
| | compared to a patient who does not adhere to the |
| | treatment at all. |
| History of relapse after | History of relapse after treatment is a relevant risk factor, |
| treatment | particularly in patients more prone or predisposed to acne |
| | scarring compared with others. |
| Location of acne lesions | In general, atrophic scars are most likely to occur on the |
| | face, often localized to cheek, temples, forehead and the |
| | mandibles. The inflammatory lesions can be quite |
| | persistent in the temples. In addition, due of the thin layer |
| | of skin in the temples it is susceptible to ineffective skin |
| | repair which further adds to the deep scarring. |
| Skin preparation | When poorly executed, skin preparation is a risk factor for |
| (comedone/cyst removal) | atrophic scarring. Poor surgical technique and use of large |
| | instruments in acne extractions escalate chances of |
| | scarring. Nevertheless, appropriate skin preparation |
| | improves acne both in terms of duration and severity. |
| Patient history of scarring | The likelihood of history of non-acne-related scarring |
| (not related to acne) | being a risk factor for acne atrophic scarring is debatable. |
| | It could be argued that this could be a risk factor, since this |
| | identifies the category of patients who are genetically |
| | prone to scarring more than others. However, not all |
| | patients who have varicella scars and acne have atrophic |
| | scars due to acne. |
| Treatment type | The type of treatment followed by the patient in the past is |
| | not attributed to formation of scars, at least, not so much |
| | as a delay in seeking treatment for significant long- |
| | standing acne condition. Recurrent damage to the skin as |
| | a result of a delay in seeking medical attention coupled |
| | with ineffective treatments is conducive to scarring. |
| Seborrheic skin type | Seborrheic skin type is unlikely to be a relevant risk factor |
| | for developing acne scars. At most, it leads to more severe |
| | acne. |
| Ethnicity | There is a weak consensus that ethnicity is a relevant risk |
| | factor, but this is based only on anecdotal evidence. |
| Gender | Gender is not an appropriate risk factor for acne atrophic |
| | scarring. |
| Smoking | Smoking is not a relevant risk factor for scarring. Some |
| | smokers do scar but the link with smoking is currently |
| | hypothetical. |
| Geographical location | There is insufficient scientific or anecdotal evidence to |
| | adequately confirm environmental factors or habitat as a |
| | relevant risk factor given the complex nature of |
| | interactions possible |
| Body mass index | In general, according to experiential evidence high BMI is |
| | not a risk-factor for atrophic acne scars. |

Supplementary Material 3:

Supplementary material related to the secondary data analysis to develop and validate the tool

Overview

Data from a cross-sectional survey conducted in Brazil, France and the USA in 2012 including patients with active acne seen during a dermatology consultation was used to develop and validate the tool. A cross-validation approach was conducted using 1,123 patients, for which the four risk factors had been collected. A random subset of 898 patients (80%) was used to develop the tool, and a random subset of 225 patients (20%) was used to validate the tool.

Tool Development

A logistic regression was conducted to assess the impact of each risk factor on the probability of developing atrophic acne scars. An optimal probability threshold of 0.31 was determined using the ROC (Receiver Operating Characteristic) curve methodology on the basis of false and true negative rates. This threshold is a trade-off between the sensitivity and the specificity of the tool. Patients reporting a predicted probability higher than 0.31 were categorized as having a “higher risk” of developing atrophic acne scars. Patients reporting a predicted probability lower than 0.31 were categorized as having a “lower risk” of developing atrophic acne scars.

This threshold was converted into a score based on a logit transformation. Each item of the tool was associated with the score reported in Table 4. The total score was the sum of each item-related score and ranges from 0 to 20. The threshold to discriminate between lower and higher risk of developing atrophic scars was estimated at 3.95.

Table validation
Findings from the cross-validation are reported in FIG. 1.
Results of the cross-validation

| | | Patients | |
|---|---|---|---|
| | Patients with | without acne | |
| | acne scars | scars | Total |
| Patients categorized as | True Positive | False positive | 157 |
| higher risk of developing | n = 88 (82%) | n = 69 (57%) | |
| scars | | | |
| Patients categorized as | False Negative | True negative | 71 |
| lower risk of developing | n = 19 (18%) | n = 52 (43%) | |
| scars | | | |
| Total | 107 | 121 | 228 |

List of Predicted Probabilities with Associated Rates

| | Proportion of | | | | | | | |
|---|---|---|---|---|---|---|---|---|
| Threshold of | patients | True | False | | True | | | |
| predicted | predicted as | positive | negative | False positive | negative | | | |
| probabilities | “High risk” | rate | rate | rate | rate | Correct | Incorrect | Outcome |
| 12% | 94% | 97% | 3% | 90% | 10% | 51% | 49% | |
| 17% | 94% | 97% | 3% | 90% | 10% | 51% | 49% | |
| 20% | 69% | 82% | 18% | 57% | 43% | 61% | 39% | |
| 23% | 69% | 82% | 18% | 57% | 43% | 61% | 39% | Selected |
| | | | | | | | | threshold |
| 28% | 64% | 78% | 22% | 52% | 48% | 62% | 38% | |
| 31% | 61% | 76% | 24% | 48% | 52% | 64% | 36% | |
| 36% | 37% | 56% | 44% | 19% | 81% | 69% | 31% | Optimal |
| | | | | | | | | threshold |
| 38% | 36% | 56% | 44% | 19% | 81% | 69% | 31% | |
| 46% | 33% | 50% | 50% | 19% | 81% | 66% | 34% | |
| 48% | 33% | 49% | 51% | 19% | 81% | 66% | 34% | |

Supplementary Material 4.

Supplementary Material Related to the Qualitative Assessment

| | | | | Answers | | | | | | |
|---|---|---|---|---|---|---|---|---|---|---|
| | | | | | Family | | | | | |
| | | | | | history | | | | | |
| | | Scar assessment | | Worst | of | | Picking | | Outcome | |
| | Skin | Self- | Dermatologist | acne | acne | Duration | and | Score | Tool | Outcome |
| Interviewees | tone* | assessment | assessment | ever | scars | of acne | squeezing | Score | outcome | review |
| 1 | II | No | No | Photo. 1 | Yes | <1 y | Sometimes | 4.60 | Higher | False |
| | | | | | | | | | risk | positive |
| 2 | IV | Yes | No | Photo 1 | No | >1 y | Never | 3.46 | Lower | Appropriate |
| | | | | | | | | | risk | |
| 3 | I | Yes | Yes | Photo 3 | Yes | >1 y | All the time | 11.11 | Higher | Appropriate |
| | | | (minimal/mild) | | | | | | risk | |
| 4 | III | No | No | Photo 1 | No | <1 y | Sometimes | 0 | Lower | Appropriate |
| | | | | | | | | | risk | |
| 5 | III | No | No | Photo 2 | Yes | >1 y | Frequently | 11.11 | Higher | False |
| | | | | | | | | | risk | positive |
| 6 | II | Yes | Yes | Photo 3 | Yes | >1 y | Sometimes | 8.06 | Higher | Appropriate |
| | | | (minimal/mild) | | | | | | risk | |
| 7 | IV | Yes | No | Photo 2 | No | >1 y | Sometimes | 3.46 | Lower | Appropriate |
| | | | | | | | | | risk | |
| 8 | VI | No | No | Photo 2 | Yes | >1 y | Never | 8.06 | Higher | False |
| | | | | | | | | | risk | positive |
| 9 | V | Yes | Yes | Photo 3 | Yes | >1 y | All the time | 11.11 | Higher | Appropriate |
| | | | (moderate) | | | | | | risk | |
| 10 | I | No | No | Photo 2 | Yes | >1 y | Rarely | 8.06 | Higher | False |
| | | | | | | | | | risk | positive |

*according to the Fitzpatrick scale

Any numbers expressing quantities of ingredients, constituents, reaction conditions, and so forth used in the specification are to be understood as being modified in all instances by the term “about.” Notwithstanding that the numerical ranges and parameters setting forth, the broad scope of the subject matter presented herein are approximations, the numerical value set forth are indicated as precisely as possible. Any numerical value, however, may inherently contain certain error or inaccuracies as evident from the standard deviation found in their respective measurement techniques. None of the features recited herein should be interpreted as invoking 35 U.S.C. § 112(f), or pre-AIA ¶6, unless the term “means” is explicitly used.

Although the present specification has been described in connection with preferred embodiments thereof, it will be appreciated by those skilled in the art that additions, deletions, modifications, and substitutions not specifically described may be made without departing from the spirit and scope of the disclosure.

### REFERENCES CITED

1. Gollnick, H.; Finlay, A.; Shear, N.; and Acne, G. A. (2008). “Can we define acne as a chronic disease? If so, how and when?” Am J Clin Dermatol., 9(5), 279-84.
2. Dréno, B.; Poili, F. (2003). “Epidemiology of Acne,” Dermatology 206(1), 7-10.
3. Bhate, K.; Williams, H. C. (2012). “Epidemiology of acne vulgaris,” British Journal of Dermatology, 168(3), 474-485.
4. Barnes, L.; Levender, M.; Fleiscer, A.; Feldman, S. (2012). “Quality of Life Measures for Acne Patients.” Dermatol Clin. 30(2), 205-354.
5. Tasoula, E.; Chalikias, J.; Danopoulou, I.; Rigopoulos, D.; Gregoriou, S.; Lazarou, D.; Katsambas, A. (2012). “The impact of acne vulgaris on quality of life and psychic health in young adolescents in Greece. Results of a population survey.” An Bras Dermatol, 87(6), 862-9.
6. Newton, J.; Mallon, E.; Klassen, A.; Ryan, T.; Finlay, A.; (1997). “The effectiveness of acne treatment: an assessment by patients of the outcome of therapy.” Br J Dermatol, 137(4), 563-7.
7. Gollnick, H. P.; Bettoli, V.; Lambert, J.; Araviiskaia, E.; Binic, I.; Dessinioti, C.; Galadari, I.; Ganceviciene, R.; Ilter, N.; Kaegi, M.; Kemeny, L.; López-Estebaranz, J. L.; Massa, A.; Oprica, C.; Sinclair, W.; Szepietowski, J. C.; Dréno, B. (2016) “A census-based practical and daily guide for the treatment of acne patients.” JEADV 30(9), 1480-90.
8. Thiboutout, D.; Gollnick, H.; Bettoli, V.; Dréno, B.; Kang, S.; Leyden, J. J.; Shalita, A. R.; Torres Lazada, V.; Berson, D.; Finlay, A.; Goh, C. L.; Herane, M. I.; Kaminsky, A.; Kubba, R.; Layton, A.; Miyachi, Y.; Perez, M.; Piquero Martin, J.; Ramos-e-Silva, M.; See, J. A.; Shear, N.; Wolf Jr., J. (2009). “New insights into the management of acne: An update form the Global Alliance to Improve Outcomes in Acne Group” J Am Acad Dermatol, 60(Suppl. 5), S1-50.
9. Zaenglein, A. L.; Pathy, A. L.; Schlosser, B. J.; Alikhan, A.; Baldwin, H. E.; Berson, D. S.; Bowe, W. P.; Graber, E. M.; Harper, J. C.; Kang, S.; Keri, J. E.; Leyden, J. J.; Reynolds, R. V.; Silverberg, N. B.; Stein Gold, L. F.; Tollefson, M. M.; Weiss, J. S.; Dolan, N. C.; Sagan, A. A.; Stern, M.; Boyer, K. M.; Bhushan, R. (2016) “Guidelines of care for the management of acne vulgaris” J Am Acad Dermatol, 74(5), 945-73.
10. Layton, A. M.; Henderson, C.; Cunliffe, W. (1994) “A clinical evaluation of acne scarring and its incidence.” Clin Exp Dermatol, 19(4), 303-8.
11. Dréno, B.; et al. (2014) P024. 23rd EADV Congress. Amsterdam, The Netherlands.
12. Bourdes, V.; Faure, C.; Petit, L.; Bettoli, V.; Bissonnette, R.; Dréno, B; Martel, P. (2015) “Natural History of Acne Lesions and Atrophic Acne Scars Within A 6-Month Study Period.” WCD. Vancouver, Canada.
13. Chuah, S. Y.; Goh, C. L. (2015) “The impact of post-acne scars on the quality of life among young adults in Singapore.” Journal of Cutaneous and Aesthetic Surgery, 8(3), 153-8.
14. Fried, R.; Werschler, P.; Berson, D.; Del Rosso, J. (2015). “The psychosocial impact of post-acne scarring.” The Dermatologist, 23(9).
15. Czemielewski, J.; Dréno, B.; Layton, A.; Bettoli, V.; Torrez, V.; Kang, S.; Acne, O. B. (2015). “Evaluation of the prevalence, risk factors, clinical characteristics and burden of acne scars among active acne patients in Brazil, France, and the USA.” World Congress of Dermatology. Vancouver, Canada.
16. Dréno, B.; Tan, J.; Kang, S.; Rueda, M.-J.; Torres, Lozada, V.; Bettoli, V.; Layton, A. (2016) “How people with facial acne scars are perceived in society: an online survey.” Dermatol The (Heidelb), 2, 207-18.
17. Munavalli, G.; Smith, S.; Maslowski, J.; Weiss, R. (2013). “Successful treatment of depressed, distensible acne scars using autologous fibroblasts: A multi-site, prospective, double blind, placebo-controlled clinical trial. Dermatologic Surgery, 39(8), 1226-36.
18. Rudnick, A.; Waibel, J. (2015) “Current trends and future considerations in scar treatment.” Seminars in Cutaneous Medicine and Surgery, 34(1), 13-16.
19. Hsu, C.-C.; Sanford, B. (2007). “The Delphi Technique: Making Sense of Consensus. Practical Assessment, Research & Evaluation, 12(10).
20. Thangaratinam, S., & Rdeman, C. (2005). The Delphi Technique. Royal College of Obstetricians and Gynecologists, 7, 120-125.
21. Hajian-Tilaki, K. (2013). Receiver Operating Characteristic (ROC) Curve Analysis for Medical Diagnostic Test Evaluation. Caspian J Intern Med, 4(2), 627-35.
22. Williams, L., Shors, A., Barlow, W., Solomon, C., & White, E. (2011). Identifying Persons at Highest Risk of Melanoma Using Self-Assessed Risk Factors. J Clin Exp Dermatol Res, 2(6).
23. Gillespie, H., Watson, T., Emery, J., Lee, A., & Murchie, P. (2011). A questionnaire to measure melanoma risk, knowledge and protective behavior: Assessing content validity in a convenience sample of Scots and Australians. BMC Medical Research Methodology, 11(123).
24. Quéreux, G., Nguyen, J.-M., Volteau, C., Lequeux, Y., & Dréno, B. (2010). Creation and test of a questionnaire for self-assessment of melanoma risk factors. European Journal of Cancer Prevention, 19(1), 48-54.
25. Basak, A., & Zaenglein, A. (2013). Acne and Its Management. Pediatrics in Review, 34(11), 479-97.
26. Holland, D., & Jeremy, A. (2005). The Role of Inflammation in the Pathogenesis of Acne and Acne Scarring. Semin Cutan Med Surg., 24(2), 79-83.
27. Fitzpatrick, T. (1988). The validity and practicality of sun-reactive skin types i through vi. Archives of Dermatology, 124(6), 869-71.

What is claimed is:

1. A method of detecting and treating an individual subject having an increased risk for acne scarring, the method comprising:
(a) measuring an increased risk factor for acne scarring in the individual subject in need thereof,
(b) diagnosing the subject with an increased risk for acne scarring based on the measured increased risk factor for acne scarring, and
(c) administering an effective amount of an anti-acne active to the subject.

2. The method according to claim 1, wherein the increased risk factor for acne scarring is quantified by at least one risk factor.

3. The method according to claim 1, wherein the at least one risk factor is at least one of a severity of acne, a family history of acne scarring, a duration of time of acne, a squeezing and picking of acne lesion behavior, and combinations thereof.

4. The method according to claim 1, wherein the subject is diagnosed with an increased risk for acne scarring when the subject scores at least 3.95 points according to the following scale:
(i) almost clear acne—0 points,
(ii) mild acne—0 points,
(iii) moderate acne—0 points,
(iv) severe acne—8.90 points,
(v) very severe acne—8.90 points,
(vi) no family history of acne scarring—0 points,
(vii) a family history of acne scarring—4.60 points,
(viii) a duration of acne of ≤1 year—0 points,
(ix) a duration of acne of >1 year—3.46 points,
(x) never squeeze and pick acne lesions—0 points,
(xi) rarely squeeze and pick acne lesions—0 points,
(xii) sometimes squeeze and pick acne lesions—0 points,
(xiii) frequently squeeze and pick acne lesions—3.05 points, and
(ix) all the time squeeze and pick acne lesions—3.05.

5. The method according to claim 1, wherein the subject measures the increased risk factor for acne scarring.

6. The method according to claim 1, wherein the subject diagnoses the increased risk factor for acne scarring.

7. The method according to claim 1, wherein the anti-acne active is a topical anti-active, an oral anti-acne active, or a combination thereof.

8. The method according to claim 1, wherein the anti-acne active is a retinoid, benzoyl peroxide, or a mixture thereof.

9. The method according to claim 8, wherein the retinoid is adapalene.

10. The method according to claim 8, wherein the retinoid is administered in a pharmaceutical or cosmetic composition comprising 0.1° A to 0.3%, by weight relative to the total weight of the composition, of the retinoid.

11. The method according to claim 1, wherein the anti-acne active is benzoyl peroxide.

12. The method according to claim 1, wherein the administration comprises administering a pharmaceutical or cosmetic composition comprising 2% to 10% by weight, relative to the total weight of the composition, of benzoyl peroxide.

13. The method according to claim 12, wherein the pharmaceutical or cosmetic composition comprises 2.5% to 5% by weight, relative to the total weight of the composition, of benzoyl peroxide.

14. The method according to claim 1, wherein the administration comprises administering a pharmaceutical or cosmetic composition comprising 2% to 10% by weight, relative to the total weight of the composition, of benzoyl peroxide and from 0.1% to 0.3% by weight, relative to the total weight of the composition, of adapalene.
